(19) United States
(12) Patent Application Publication
(10) Pub. No.: US 2011/0236503 A1
(43) Pub. Date: Sep. 29, 2011

(54) Topical Skincare Composition

(75) Inventor: Theodore A. Kalli, San Diego, CA (US)

(73) Assignee: COSMALABS INTERNATIONAL, LLC, San Diego, CA (US)

(21) Appl. No.: 12/729,566

(22) Filed: Mar. 23, 2010

(51) Int. Cl. A61K 33/40; A61P 17/10

(52) U.S. Cl. 424/616

(57) ABSTRACT

A composition and method for treating dermatological disorders (e.g., acne) is disclosed. In accordance with an embodiment, an antimicrobial composition includes a therapeutically effective amount of benzoyl peroxide and a therapeutically effective amount of hydrogen peroxide, where the combination of the benzoyl peroxide and hydrogen peroxide produces a synergistic effect, thereby enhancing the properties of both the benzoyl peroxide and hydrogen peroxide. In accordance with another embodiment, a skincare composition suitable for topical application to the skin includes from 1.0 to 10.0% by weight of benzoyl peroxide, and from 0.5 to 3.0% by weight of hydrogen peroxide, where the composition is substantially free of other therapeutic agents selected from the group consisting of anti-microbial agents, anti-bacterial agents, anti-viral agents, anti-fungal agents, and anti-inflammatory agents.

### FIELD OF THE INVENTION

The present invention relates, in general to skincare compositions, more particularly to compositions effective in the treatment of acne vulgaris and to methods of treatment of the skin that involve the topical application of such compositions.

### BACKGROUND OF THE INVENTION

Human skin is a composite material of the epidermis and the dermis. The topmost part of the epidermis is the stratum corneum. This layer is the stiffest layer of the skin, as well as the one most affected by the surrounding environment. Below the stratum corneum is the internal portion of the epidermis. Below the epidermis, the topmost layer of the dermis is the papillary dermis, which is made of relatively loose connective tissues that define the micro-relief of the skin. The reticular dermis, disposed beneath the papillary dermis, is tight, connective tissue that is spatially organized. The reticular dermis is also associated with coarse wrinkles. At the bottom of the dermis lies the subcutaneous layer.

The principal functions of the skin include protection, excretion, secretion, absorption, thermoregulation, pigmentogenesis, accumulation, sensory perception, and regulation of immunological processes. These functions are detrimentally affected by, for example, dryness, yeast, and structural changes in the skin, such as due to aging and excessive sun exposure.

Acne vulgaris (acne) is a chronic inflammatory condition of the pilosebaceous units of the skin, which is particularly prevalent in adolescents. The condition generally causes the formation, on the skin, of comedones, red papules, pustules and sometimes cysts. This is unsightly and furthermore, if untreated, acne can lead to scarring of the skin. The major causes of acne are thought to be an increase in sebum production, an increased presence of Propionibactedum acnes (P. acnes), blockage of the pilosebaceus duct and the production of inflammation.

Various pharmaceuticals have been used for the treatment or prevention of skin conditions, including skin cleansing compositions. Some of these compositions are discussed below.

Benzoyl peroxide is known to be effective in the treatment of acne. Benzoyl peroxide is clinically effective because of its bactericidal activity against P. acne as well as through its mild keratinolytic effect.

It has now been found that an improved topical acne treatment can be achieved by combining benzoyl peroxide with hydrogen peroxide.

Hydrogen peroxide has also been employed in cleansing compositions for topical application to the skin. However, hydrogen peroxide is generally regarded merely as a disinfectant, and has not been employed as an active agent in the treatment of acne.

Surprisingly, it has now been found that skincare compositions comprising therapeutically effective concentrations of both benzoyl peroxide and hydrogen peroxide are effective in the treatment of acne without requiring the presence of an anti-microbial agents, anti-bacterial agents, anti-viral agents, anti-fungal agents, or anti-inflammatory agents. In particular, the combination of benzoyl peroxide and hydrogen peroxide is believed to have valuable therapeutic properties in reducing the presence of P. acnes on the skin, especially resulting from the oxidation effect of the hydrogen peroxide.

### SUMMARY OF THE INVENTION

The present invention provides a composition and method for treating acne. The method includes topically administering to a human patient in need of such an acne treatment, a safe and therapeutically effective amount of a composition including at least benzoyl peroxide and hydrogen peroxide.

In accordance with an embodiment, an antimicrobial composition includes a therapeutically effective amount of benzoyl peroxide and a therapeutically effective amount of hydrogen peroxide, where the combination of the benzoyl peroxide and hydrogen peroxide produces a synergistic effect, thereby enhancing the properties of both the benzoyl peroxide and hydrogen peroxide.

In accordance with another embodiment, a skincare composition suitable for topical application to the skin includes from 1.0 to 10.0% by weight of benzoyl peroxide, and from 0.5 to 3.0% by weight of hydrogen peroxide, where the composition is substantially free of other therapeutic agents selected from the group consisting of anti-microbial agents, anti-bacterial agents, anti-viral agents, anti-fungal agents, and anti-inflammatory agents.

### DETAILED DESCRIPTION OF PREFERRED EMBODIMENTS

After reading this description it will become apparent to one skilled in the art how to implement the invention in various alternative embodiments and alternative applications. However, all the various embodiments of the present invention will be described herein, it is understood that these embodiments are presented by way of an example only, and not limitation. As such, this detailed description of various alternative embodiments should not be construed to limit the scope or breadth of the present invention as set forth below.

### Benzoyl Peroxide—[C₆H₅C(O)]₂O₂

Benzoyl peroxide was first used for treatment of acne in 1979 as a potent topical bactericidal agent. Benzoyl peroxide reduces the population of P. acnes by generating reactive oxygen species (“ROS”) within the sebaceous follicle.

Benzoyl peroxide is effective against inflammatory and non-inflammatory lesions and treats acne bacteria as early as 5 days after initiation of treatment. Benzoyl peroxide is offered in a wide variety of preparations (e.g., cleansers, lotions, creams, gels and pads) and reduction in P. acnes is effective in leave-on and wash-off products.

When compared with other topical antimicrobials, a topical composition having 5% benzoyl peroxide was found to be at least as effective for acne as erythromycin and clindamycin formulations. Erythromycin and clindamycin are currently commonly used for the treatment of acne. However, propionibacterial resistance to these antibiotics is a growing concern because of its high resistance. In contrast, benzoyl peroxide has the ability to prevent or eliminate the development of P. acnes resistance.

In the United States, the typical concentration for benzoyl peroxide is 2.5% to 10% for both prescription and over the counter preparations that are used in treatment for acne.

### Hydrogen Peroxide—H₂O₂

Hydrogen peroxide generally decomposes exothermically into water and oxygen gas according to the following reaction:

2H₂O₂→2H₂O+O₂ Equation 1

Hydrogen peroxide is naturally produced in organisms as a byproduct of oxygen metabolism. Nearly all living things possess enzymes known as peroxidases, which harmlessly and catalytically decompose low concentrations of hydrogen peroxide to water and oxygen.

When hydrogen peroxide contacts the skin, it generally breaks down into water and oxygen due to the presence of the enzyme Catalase. The free oxygen thus obtained penetrates deep down into the pores of the skin and reach the source of the acne. As a result, the bacteria get killed as they cannot survive in presence of oxygen. On the other hand, the water helps to open up the clogged pores.

Hydrogen peroxide is generally recognized as safe (“GRAS”) as an antimicrobial agent, an oxidizing agent, and for other purposes by the Food and Drug Administration (“FDA”). Hydrogen peroxide has been used as an antiseptic and anti-bacterial agent for many years due to its oxidizing effect. While its use has decreased in recent years with the popularity of readily available over the counter products, it is still used by many hospitals, doctors and dentists.

### Compositions

Surprisingly, it has now been found that skincare compositions comprising therapeutically effective concentrations of both benzoyl peroxide and hydrogen peroxide are effective in the treatment of acne without requiring the presence of either an anti-microbial or an anti-inflammatory therapeutic agent. In particular, the combination of benzoyl peroxide and hydrogen peroxide is believed to have valuable therapeutic properties in reducing the presence of P. acnes on the skin, especially resulting from the oxidation effect of the hydrogen peroxide.

Without wishing to be bound by any particular theory, in the present compositions, instead of the oxygen being released on the surface of the skin and escaping to the atmosphere, as with aqueous hydrogen peroxide, the oxygen penetrates the skin. This is due to the unique formulations, in which an oil phase in an emulsion creates a barrier to the oxygen, and the skin becomes the path of least resistance. When hydrogen peroxide changes from a liquid to a gas (which may be instantaneous), it increases in volume 22.4 times. This increase in volume is believed to causes the pressure and why the hydrogen peroxide penetrates the skin.

Furthermore, it is believed that the oxygen is a gas only during this instantaneous reaction. When it penetrates the skin, it is dissolved in the extracellular water and in the capillary plasma, as molecular oxygen (gas) can only exist in the lungs.

The presence of oxygen may be measured by medical equipment such as a Cutaneous Oxygen Monitor (e.g., Cutan.PO₂ Monitor 820). The increase of moisture content may be measured with a Scalar Moisture Checker (e.g., MY-808S). Measurements before and after using compositions in accordance with embodiments of the invention show an increase in both cutaneous oxygen and moisture.

As used herein, the term ‘about’ will be understood by persons of ordinary skill in the art and will vary to some extent on the context in which is used. If there are uses of the term which are not clear to persons of ordinary skill in the art given the context in which it is used, ‘about’ will mean up to plus or minus 10% of the particular term.

The term ‘pharmaceutically acceptable’, as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term ‘safe and effective’, as used herein, means a concentration of an active ingredient or an amount of a composition, that is sufficient enough to significantly and positively modify the condition to be treated but low enough to avoid serious side effects, within the scope of sound medical advice.

All percentages referred to in this specification are percentages by weight of the total composition unless otherwise indicated.

The provision of an efficacious acne treatment composition containing benzoyl peroxide and hydrogen peroxide without the anti-microbial and/or anti-inflammatory agent is unexpected. Furthermore, reducing the number of active ingredients in a composition is of significant advantage as it facilitates formulation and also manufacturing processes. The combination of these two ingredients also allows the preparation of a wide variety of non-irritant, stable and cosmetically acceptable therapeutic compositions, including, but not limited to, compositions comprising detergent systems. The combination also provides efficacious compositions for topical application which may be adapted either for leaving on the skin after being applied thereto or for being rinsed off after application.

In accordance with some embodiments, benzoyl peroxide and hydrogen peroxide are the sole active ingredients having a therapeutic effect in the topical treatment of acne. In one embodiment of the invention, benzoyl peroxide and hydrogen peroxide are the sole active ingredients in the composition.

Benzoyl peroxide is preferably incorporated into the composition according to the invention as gel or gel solution. As used herein, unless the context requires otherwise, any and all references to benzoyl peroxide should be taken to encompass references to the peroxide and to gel or gel solution forms.

The concentration of benzoyl peroxide in the composition according to the invention is preferably at least 1.0% by weight, more preferably at least 1.5% and most preferably at least 2.0% by weight. The concentration of benzoyl peroxide is preferably less or equal to about 10% by weight, more preferably less than 8% by weight, and most preferably less than 6% by weight. The concentration of benzoyl peroxide may therefore fall in the range 1.0% to 10% by weight, more preferably 1.5% to 8%, and most preferably 2.0% to 5.0% by weight.

The composition most preferably comprises hydrogen peroxide. Alternatively, the composition may comprise a compound that, in use, is capable of generating hydrogen peroxide. An example of the latter class of compound is an adduct such as urea peroxide (e.g., carbamide peroxide).

The concentration of hydrogen peroxide in the composition according to the invention is preferably at least 0.5% by weight. The concentration of hydrogen peroxide is preferably less than about 3% by weight, more preferably less than 2.5% by weight. The concentration of hydrogen peroxide may therefore fall within the range 0.5% to 3% by weight, more preferably 1% to 3%, and most preferably 1% to 2% by weight.

In accordance with some embodiments, the ratio of benzoyl peroxide to hydrogen peroxide in a composition according to the invention is in the range from 20:1 to 1:3 parts by weight, preferably from 8:1 to 1:2 parts by weight, and most preferably from 5:1 to 1:1 parts by weight.

The composition is preferably prepared with a pH in the range 5.5 to 7.5, more preferably 6.0 to 7.0, and particularly a pH in the range 6.2 to 6.8.

The composition according to the invention may be formulated in numerous forms. However, the composition may often take the form of an aqueous or oily solution or dispersion or emulsion or a gel. An emulsion may be an oil-in-water emulsion or a water-in-oil emulsion.

### Example

This example is to prepare a topical composition containing 8% benzoyl peroxide and 2% hydrogen peroxide. The aqueous composition in accordance with embodiments of the invention was prepared as follows:

| | | Amount (weight |
|---|---|---|
| | Component | percentage) |
| Phase I | Petrolatum | ≦4.5% |
| | Cetearyl Alcohol | 5.0% |
| | Mineral Oil | 6.0% |
| | Polysorbate 60 | 3.2% |
| | Tocopherol | 0.8% |
| Phase II | Purified Water | ≦100% |
| | Glycerin | 5% |
| | Potassium Sorbate | 0.15% |
| | Tetrasodium EDTA | 0.25% |
| | Phenoxyethanol | 0.08% |
| | Ethylhexyglycerin | 0.02% |
| Phase III | Benzoyl Peroxide | 1.0-10.0% |
| Phase IV | Hydrogen Peroxide | 0.5-3.0% |

In a first embodiment, each of the phases is prepared separately. For example, the ingredients in Phase I are combined and heated to about 70° C. In some embodiments, Phase I ingredients are combined and heated to between about 50° C. and 90° C.

In a separate vessel, the ingredients in Phase II are combined and heated to about 70° C. In some embodiments, Phase II ingredients are combined and heated to between about 50° C. and 90° C.

It should be appreciated that Phase I and Phase II each make up separate parts of a base cream. Thereafter, ingredients from Phase I are combined with ingredients from Phase II. This mixture of Phase I and Phase II is agitated slowly, such as by stirring, and allowed to cool. In one embodiment, the mixture of Phase I and Phase II is cooled to about 62° C. In some embodiments, the mixture of Phase I and Phase II is cooled to between about 50° C. and 70° C.

The mixture of Phase I and Phase II is preferably homogenized for a predetermined time, such as between 5 and 20 minutes with sufficient agitation, such as stirring at between 3,000 to 5,000 revolutions per minute (RPM). In one embodiment, the mixture is homogenized for about 12 minutes at 3,800 RPM.

Thereafter, the mixture of Phase I and Phase II is allowed to cool to about 30° C., while continuing to agitate the mixture slowly. Upon reaching the cooled temperature of about 30° C., Phase III is added to the mixture of Phase I and Phase II. This mixture of Phase I, II, and III is preferably homogenized for a predetermined time, such as between 3 and 15 minutes with sufficient agitation, such as stirring at between 2,000 to 4,000 RPM. In one embodiment, the mixture is homogenized for about 7 minutes at 3,400 RPM.

The mixture of Phase I, II and III is allowed to further cool to about 28° C., while continuing to agitate the mixture slowly. Phase IV is thereafter added to the mixture if Phase I, II, and III. This mixture of Phases I, II, III and IV is slowly agitated (e.g., less than 3,400 RPM) and allowed to cool to about 21° C.

Upon reaching 21° C., agitation is suspended. The mixture's pH is then taken. In one embodiment, a preferred pH is between about 6.2 and 6.8. If the pH falls outside this window (e.g., 6.2-6.8) a neutralizer may be used to bring the pH into this preferred window.

Preferably, the resultant mixture is smooth. If the mixture is not smooth, various additional steps, such as further agitation and/or addition of other ingredients may be performed. The resultant mixture may, in some embodiments, have a viscosity of between about 39,000 and 43,000 centipoise (cP). Additionally, in some embodiments, the resultant mixture may have a density of between about 0.995 and 1.003 g/cm³.

Compositions made in accordance with embodiments of the present invention enjoy a number of benefits. For example, having this unique combination of hydrogen peroxide and benzoyl peroxide enhances the beneficial properties (e.g., synergistic effect) of both ingredients for the treatment of acne. Additionally, the increase in the moisture content of the skin (e.g, caused by hydrogen peroxide penetrating the skin) aids in the feeling of dryness, normally associated with benzoyl peroxide and other acne products.

Additionally, in some embodiments, other ingredients may be included in the present compositions. For example, Phase III may additionally include or more of the following: purified water (aqua), mineral oil, petrolatum, glycerin, cetearyl alcohol, tetrasodium, potassium sorbate, phenoxylethanol, ethylhexylglycerin and tocopherol. The additional ingredients may have the following functionality: the purified water serves as a solvent, the mineral oil serves as a stabilizer, the petrolatum serves as an emulsifier, the glycerin serves as a humectant, the cetearyl alcohol serves as an emulsifier, the tetrasodium serves as a stabilizer, the potassium sorbate serves as a preservative, the phenoxylethanol serves as a preservative, the ethylhexylglycerin serves as a preservative, and the tocopherol serves as a vitamin.

In accordance with an embodiment, these additional ingredients in Phase III are inactive ingredients. Consequently, in accordance with a preferred embodiment, the only active ingredients in a resulting composition are benzoyl peroxide and hydrogen peroxide.

### Directions for Use

In the morning and/or evening, cleanse the area to be treated. Rinse thoroughly and dry the area. Apply a small amount of the cream (e.g., composition) to the affected areas. Do not massage in. After two to three minutes, use gentle circular movements to disperse the product over the applied area.

Various preferred embodiments of the invention have been described in fulfillment of the various objects of the invention. It should be recognized that these embodiments are merely illustrative of the principles of the invention. Numerous modifications and adaptations thereof will be readily apparent to those skilled in the art without departing from the spirit and scope of the present invention.

For example, while the compositions described herein have been discussed as a topical acne treatment, it should be appreciated that the compositions may be used for the treatment of many dermatological disorders. This is because the compositions described herein are general antimicrobial formulations.

Formulations according to embodiments of the invention are preferably suitable for, and more preferably adapted for, topical administration to human or animal, in particular human, skin. Formulations may also be suitable for, or adapted for, topical administration to other epithelia such as the nares, scalp, ears, eyes, vagina and oral cavity. Formulations may take the form of a lotion, cream, ointment, foam, paste or gel or any other physical form known for topical administration, including for instance a formulation which is, or may be, applied to a carrier such as a sponge, swab, brush, tissue, skin patch, dressing or dental fiber to facilitate its topical administration. Formulations may take the form of a nasal spray or of eye or ear drops. Formulations may be intended for pharmaceutical (which includes veterinary) use, for example to treat skin infections or as a prophylactic against infections such as MRSA, and/or for cosmetic or other non-medical care purposes (for example, for general hygiene or cleansing).

It is to be understood that the following claims are intended to cover all of the generic and specific features of the invention herein described and all statements of the scope of the invention, which might be said to fall there between.

Particularly, it is to be understood that in the claims, ingredients or compounds recited in the singular are intended to include compatible mixtures of such ingredients wherever the sense permits.

What is claimed is:

1. An antimicrobial composition comprising:
a therapeutically effective amount of benzoyl peroxide; and
a therapeutically effective amount of hydrogen peroxide;
wherein the combination of the benzoyl peroxide and hydrogen peroxide produces a synergistic effect, thereby enhancing the properties of both the benzoyl peroxide and hydrogen peroxide.

2. The antimicrobial composition of claim 1, wherein a therapeutically effective amount of benzoyl peroxide is up to 10% w/v.

3. The antimicrobial composition of claim 3, wherein the therapeutically effective amount of benzoyl peroxide is between 1 and 10% w/v.

4. The antimicrobial composition of claim 1, wherein a therapeutically effective amount of hydrogen peroxide is up to 3% w/v.

5. The antimicrobial composition of claim 4, wherein a therapeutically effective amount of hydrogen peroxide is between 0.5 and 3% w/v.

6. The antimicrobial composition of claim 1, wherein the composition is suitable for topical application to the skin.

7. The antimicrobial composition of claim 1, wherein the composition is effective at reducing the presence of P. acnes on the skin.

8. The antimicrobial composition of claim 7, wherein the reduction in P. acnes is caused in part by the oxidation effect of the hydrogen peroxide.

9. A skincare composition suitable for topical application to the skin, the composition comprising from 1.0 to 10.0% by weight of benzoyl peroxide, and from 0.5 to 3.0% by weight of hydrogen peroxide, wherein the composition is substantially free of other therapeutic agents selected from the group consisting of anti-microbial agents, anti-bacterial agents, anti-viral agents, anti-fungal agents, and anti-inflammatory agents.

10. The composition of claim 9, wherein the concentration of benzoyl peroxide in the composition is between 1.5 and 6% by weight.

11. The composition of claim 9, wherein the concentration of hydrogen peroxide in the composition is between 1.0 and 3% by weight.

12. The composition of claim 9, wherein the pH of the composition is in the range of 6.0 to 7.0.

13. The composition of claim 12, wherein the pH is in the range of 6.2 to 6.8.

14. The composition of claim 9, the composition further comprising one or more of the following: purified water (aqua), mineral oil, petrolatum, glycerin, cetearyl alcohol, tetrasodium, potassium sorbate, phenoxylethanol, ethylhexylglycerin and tocopherol.

15. The composition of claim 9, the composition having the form of an aqueous or oily solution or dispersion or emulsion or gel.

16. The composition of claim 9, the composition having a viscosity of between 39,000 and 43,000 cP.

17. The composition of claim 9, the composition having a density of between 0.995 and 1.003 g/cm³.

18. A method for treating a dermatological disorder comprising administering a therapeutically safe and effective amount of the composition as claimed in claim 9 to a human patient in need of treatment.

19. The method of claim 18, wherein the dermatological disorder is acne.
